(19) United States
(12) Patent Application Publication
(10) Pub. No.: US 2014/0242641 A1
(43) Pub. Date: Aug. 28, 2014

(54) METHOD FOR OBTAINING AN OPEN PHOTOTROPHIC CULTURE WITH IMPROVED STORAGE COMPOUND PRODUCTION CAPACITY

(71) Applicants: Jelmer Tamis, Delft (NL); Robert Kleerebezem, Delft (NL); Mark Cornelis Maria van Loosdrecht, Delft (NL)

(72) Inventors: Jelmer Tamis, Delft (NL); Robert Kleerebezem, Delft (NL); Mark Cornelis Maria van Loosdrecht, Delft (NL)

(73) Assignee: Technische Universiteit Delft, Delft (NL)

(21) Appl. No.: 14/233,619

(22) PCT Filed: Jul. 18, 2012
(86) PCT No.: PCT/NL2012/050515

§ 371 Date: Mar. 31, 2014

(30) Foreign Application Priority Data
Jul. 18, 2011 (NL) 2007132

(51) Int. Cl. C12N 1/12; A01G 33/00; C12P 7/62; C12P 7/64; C12P 19/04

(52) U.S. Cl. CPC C12N 1/12; C12P 7/64; C12P 19/04; C12P 7/625; A01G 33/00
USPC 435/101; 435/257.1; 435/134; 435/135; 47/1.4

(57) ABSTRACT

The invention is directed to a method for producing an open phototrophic culture with improved storage compound production capability.

In accordance with the invention, a starting culture is submitted to selective pressure, thus giving a competitive advantage to storage compound producing species, by subjecting said starting culture to a cycle of alternating dark phases and light phases and providing limitation of availability of essential growth nutrients in one or more of said light phases. The resulting culture can be used to provide storage compounds in improved yields.

The invention is directed to a method of obtaining a phototrophic culture with superior storage compound production capacity. The cultures produced in accordance with the invention can be used for producing biological storage compounds by culturing the biological storage compounds producing phototrophs contained in said cultures.

Over the last few decades an extensive amount of research has been carried out in an attempt to develop biofuels and other biobased products from sustainable resources. A variety of different biomasses from different sources have been researched for the production of different biofuels including biodiesel, bio-ethanol, biogas, bio-hydrogen, bio-oil and bio-syngas. Biofuel sources such as sugar based ethanol and palm oil (or other agrocrops such as soybean, rapeseed and sunflower) were found to have the disadvantages that they compete with food crops and impact biodiversity and nature.

The use of aquatic phototrophic microorganisms, in particular algae, is generally seen as more environmentally sound because primary production with algae can be more efficient than with higher plants. In particular the farming of algae enables higher areal yields and provides the possibility of using non-arable land or ocean.

The U.S. National Renewable Energy Labs (NREL) evaluated in the period between the late 1970s and 1990s the economic feasibility of producing biofuels from a variety of aquatic and terrestrial photosynthetic organisms (see Sheehan et al., “A look back at the U.S. Department of Energy's Aquatic Species Program-Biodiesel from Algae”, National Research Energy Laboratory (1998)). In this study nutrient starvation, such as N or Si limited growth, was used to induce a change in lipid compositions in algal cells. It was determined that biofuel production from microalgae had greater potential than terrestrial sources. However, they concluded that at that time it was not economically feasible to produce biodiesel using algal oils.

More recently the economics and quality constraints of biodiesel were discussed in an academic review paper (see Chisti Y., “Biodiesel from microalgae”, Biotechnol. Adv., 25(2007)294-306). It was concluded that the cost of growing microalgae for biofuel production needed to be drastically reduced to compete directly with traditional energy sources.

Harrison, W. G. (J. exp. mar. Biol. Ecol. 21(1976)199-209) describes a study to the metabolism in specific species of algae (Gonyaulax polyedra Stein).

Feng, D. et al. (Bioresource Technology 102(2011)6710-6716) describes a study to the lipid production metabolism in another specific species of algae (Isochysis zhangjiangensis).

All these known studies, however, focus on pure cultures (“unialgal” cultures). In fact, all research directed to optimizing production of storage compounds in algae, has been focused on finding single species with improved properties. However, the use of pure cultures has the disadvantage of high cost from pre-sterilized equipment, and problems related to infection of other species and reduced productivity due to the algae culture growing more slowly in nutrient-limited conditions.

The present inventors realized that it is much more efficient not to have to rely on sterile conditions. Using phototrophic microorganisms as a carbon source for fuel and chemicals production can reduce the production costs compared to conventional agriculture if it would be possible to provide a culture of phototrophic microorganisms with high storage compound content in relatively cheap systems such as open ponds. Desirable functionality of this culture is to contain phototrophic species that have high lipid or other storage compounds producing capacity and produce a biomass which contains a substantial amount of storage compounds.

It is therefore desirable to find a method for obtaining a phototrophic culture with superior storage compound production capacity, so that it becomes a more economically viable biofuel source.

Considering the status of the phototrophic species cultivation technology, especially the fact that high storage compound productive algal cultures have not been shown to have stable growth in open (non-sterile) ponds during many generations, it is an object of the present invention to provide a method for obtaining a stable phototrophic culture with superior storage compound production. The present inventors surprisingly found that this can be achieved by application of a selective pressure from the environment. This method is based on selective enrichment and can be used to obtain desired non-axenic cultures in non-sterile environments.

Surprisingly, it was found that by subjecting a starting culture to selective pressure with the aim of improving yield of storage compounds, results in a so-called “open” culture, that still has the desired improved product yield. An open culture is defined herein as a culture that needs not be confined to a single species, so preferably it contains two or more species. Apart from two or more different phototrophic species (such as algae), also other organisms may be present, such as bacteria, or fungi. This means that the equipment used does not have to be sterile, which is a great advantage. The culture used is thus a non-axenic culture, that may comprise more than one specie of algae. Preferably the open culture of the present invention is non-axenic.

The present invention is accordingly directed to a method for producing an open phototrophic culture with improved storage compound production capability, comprising subjecting a starting culture to selective pressure, thus giving a competitive advantage to storage compound producing species, by subjecting said starting culture to a cycle of alternating dark phases and light phases and providing limitation of availability of essential growth nutrients in one or more of said light phases.

The open culture is obtained by selecting a starting culture, comprising one or more species, in particular algae species and subjecting them to the selective pressure by subjecting the starting culture to a cycle of alternating dark phases and light phases and providing limitation of availability of essential growth nutrients in one or more of said light phases. No special measures, such as sterilization of the equipment needs to be taken. As a result other species will also start to develop in the volume holding the original starting culture, for instance because the original culture becomes “contaminated” with these other species. Thus an open culture in accordance with the invention is obtained. Surprisingly this open culture has developed such that it provides very high production of storage compounds.

Surprisingly it was found that by using selective pressure and evolution a stable open, preferably non-axenic phototropic culture can be obtained. This open culture surprisingly produces much higher storage compound contents than observed for open cultures on which the selective pressure in accordance with the invention was not exercised. In accordance with the invention it is possible to produce algae wherein the total mass of storage compound is at least 50 wt. %, preferably at least 70 wt. % and more preferably 80-90 wt. % based on the weight of the dried cell mass. Apart from storage compound content also the open culture provides superior specific productivity of storage compounds. For instance, it was found possible to obtain an increase in storage compounds of more than 40 wt. % compared to the weight of storage compounds present in the starting culture already within 7 hours of light supported cultivation: the initial storage was only 2 wt. % and after 7 hours is was 42 wt. %, which is an increase of more than a factor 20.

The term “non-axenic” culture, as used herein refers to a culture that in principle has a free exchange of biological material with its surroundings, and can constantly be invaded by all kind of new species. The specie or species that can adapt the best to the environmental conditions in the culture in terms of sustaining growth will in principle become dominant in the culture.

We use selective pressure for mixed cultures as a tool to obtain the culture functionality and reactor performance of choice. In this way there is no need for expensive equipment and energy for sterilization since infections with competitive phototrophic organisms are rendered harmless or even beneficial. Moreover and importantly long term continuous operation of the reactor system becomes possible.

Johnson et al. (Johnson, K., Jiang, Y., Kleerebezem, R., Muyzer, G. and van Loosdrecht, M. C. M. Enrichment of a mixed bacterial culture with a high polyhydroxyalkanoate storage capacity, Biomacromolecules 10 (2009) 670-676) established in their research on the production of bioplastics, such as polyhydroxyalkanoates (PHAs) from organic waste streams that these bioplastics can be produced with open mixed bacterial cultures if a suitable enrichment step based on the ecological role of PITA is used. The use of sunlight as an energy source and CO₂ as carbon source for these kinds of applications, however, opens up a new field of possibilities.

The present invention relates to a selection method based on the ecological role of storage compounds, in particular compounds comprising sugars and/or polyhydroxyalkanoates and/or lipids and/or other carbon-based compounds in a day/night cycle.

It was found that uncoupling of carbon fixation and growth in a phototrophic community can be established by feeding the phototrophic community with a limited amount of nutrients in the absence of autotrophic carbon fixation (in the dark phase). By providing nutrients in the dark period the species capable of growing on storage compounds have a competitive advantage over species that depend directly on light for growth. By full (or almost full, e.g. more than 95 wt. %) consumption of one or more essential growth nutrients during growth on storage compounds in the dark phase, no significant active biomass growth is possible in the subsequent light period. Therefore, in the light phase, substantially all carbon dioxide fixation that occurs leads to the formation of storage compound molecules in the microorganism and not to any substantial growth. Application of these conditions of repeating light phases without nutrients and dark phases with nutrients circumstances over many cycles (typically more than 5, more typically more than 10 cycles) was found to lead to a phototrophic community that is enriched with species with a superior storage compound producing capacity and the capacity to grow on storage compounds in the dark.

The method of the present invention is conceptually different from the method used by Johnson et al. mentioned above. This is because the approach of Johnson et al. is based on an overflow metabolism in which the difference between the maximum growth rate and carbon uptake of the bacteria is used as strategy for application of selective pressure. The method of the present invention, on the other hand, comprises the separation of growth and carbon uptake of phototrophs.

Surprisingly, despite the large attention for algal based methods no one has previously come up with the present approach. The advantage of this method is that it provides for a relatively simple operation and a high productivity, resulting in cost reduction compared to existing options, in particular because it is not necessary to work under sterile conditions.

The method of the invention may be followed by conventional process steps, including:
collecting the biomass;
extracting the storage compounds from the biomass; and
converting the storage compounds into a valuable product, preferably biofuel.

Without wishing to be bound by theory it is believed that organisms that somehow can perform enhanced storage compound accumulation in response to inducing time varying environmental conditions such as combined temporary nutrient depletion and day/night cycling have a competitive advantage over other species and will therefore become dominant in an open culture, essentially defining therewith the properties of this culture.

The dark phase is typically 2-72 hours, preferably 4-48 hours and more preferably 6-18 hours. There is essentially no light source present during the dark phase. The light phase is typically from 2-72 hours, preferably 4-48 hours and more preferably 6-18 hours. A light source is present during the light phase. A suitable light source may be sunlight or an artificial light source. Preferably the light source is sunlight. Preferably the method of the invention is carried out in an open pond, subjecting the open culture to sunlight during daytime while limiting nutrients and feeding nutrients during night time.

A growth limiting amount of an essential growth nutrient should be supplied in the dark phase. In this case growth limiting nutrients for phototrophic organisms may include compounds containing nitrogen, phosphorus, sulfur, molybdenum, magnesium, cobalt, nickel, silicon, iron, zinc, copper, potassium, calcium, boron, chlorine, sodium, selenium, specific vitamins and any other compounds that may be essential for biomass assimilation of phototrophic species.

The typical amount of nutrients present in a phototrophic fresh water growth medium may be according to the composition of the COMBO medium developed by Kilham, S. S., Kreeger, D. A., Lynn, S. G., Goulden, C. E. and Herrera, L., Hydrobiologia (1998) 377, 147-159. However, other phototrophic growth media known to those skilled in the art may also be suitably used. In accordance with the invention a modified version of these know media may be used, specifically with regard to the one or more omitted essential nutrients.

A preferred nutrient that can be supplied in growth limiting amounts is nitrogen. While nutrient deficiency (i.e. nitrogen deficiency) prevents growth and production of most cellular components, the production of storage compound synthesis remains possible.

Repetition of the abovementioned dynamic pattern over many day/night cycles leads to the selection of a phototrophic community with a superior storage compound production capacity.

Other preferred nutrients which may be depleted include iron, phosphorus and magnesium.

The cultivation method of the present invention is different from the prior art in that it is open and thus does not rely on pure cultures, in which no selective pressure is present.

The method of the present invention also differs from methods wherein cultures that are depleted for nutrients in order to induce storage compound production, but in which however no selective pressure in applied.

The terms “phototroph” and “phototrophic” are defined as properties of organisms that use photons as an energy source. In principle all phototrophic life forms can be used in accordance with the present invention, in particular prokaryotes (such as cyanobacteria), archaea, algae and eukaryotes. Preferably algae are used in accordance with the present invention.

Both freshwater and marine phototrophic species are suitable to be constituents of the culture to be used in accordance with the invention. Some species that may be present in the culture for this purpose include Bacillariophyceae, Chlorophyceae, Cyanophyceae, Xanthophyceaei, Chrysophyceae, Chlorella, Crypthecodinium, Schizocytrium, Nannochloropsis, Ulkenia, Dunaliella, Cyclotella, Navicula, Nitzschia, Cyclotella, Phaeodactylum, and Thaustochytrid classes and genera and other. The starting culture that is used for the present invention may contain one or more of these species and/or other species. A very suitable mixture of starting culture can be obtained by using a sample (typically 1-10 dm³) of surface water, for instance from canals, ponds, lakes, rivers, oceans, etc. As illustrated in the examples below a sample taken from a Dutch city canal is very suitable.

In accordance with the invention the open culture generally contains a mix of a number of species, which may include many more species than those mentioned above. In fact it is not even necessary to know which species are present in the open culture, since the desirable species are selected by applying the selective pressure, as explained above.

The terms “storage compound” and “microbial storage compound” include lipids, polysaccharides or other carbon-based compounds like for example polyhydroxyalkanoates. Preferably the storage compound is a lipid.

The term “lipids” includes naturally occurring fats, waxes, sterols, phospholipids, free fatty acids, monoglycerides, diglycerides and triglycerides and other hydrophobic or amphiphilic carbon based biological molecules. Free fatty acids typically have a carbon chain length from 14 to 20, with varying degrees of unsaturation. A variety of lipid derived compounds can also be useful as biofuel and may be extracted from phototrophs. These include isoprenoids, straight chain alkanes, and long and short chain alcohols, with short chain alcohols including glycerol, ethanol, butanol, and isopropanol. Preferably, the lipids are triacylglycerides (TAGs) and are synthesized in phototrophs through a biochemical process involving various enzymes such as trans-enoyl-acyl carrier protein (ACP), 3-hydroxy acyl-ACP, 3-ketoacyl-ACP, and acyl-ACO or other enzymes.

The term “polysaccharides” includes glycogen, starches and other carbohydrate polymers. Preferably the polysaccharide is glycogen.

Typically the phototrophic community is grown in an open system. An open system is defined as a system that may comprise more than one species. preferably the culture making up the phototrophic community is non-axenic. No sterilization is required and all sorts of microorganisms can enter the system. In a preferred embodiment the method of the invention is carried out under non-sterile conditions. Thus the equipment used for carrying out the invention is preferably not sterilized prior to use, which saves tremendously in costs. Open systems may be open reactors, open tanks, natural water bodies, (raceway) ponds and artificial reservoirs or other water containing open spaces. The open systems are typically fairly shallow (typically less then 1 m deep, preferably from 20 cm to 50 cm) so as to allow light to reach the majority of the phototrophs within the systems, and typically have a consistent depth to provide the maximum area for growth within the zone that is accessible to light. Preferably the open system used is an open reactor. A typical open reactor design for phototrophic organisms is a raceway pond.

The advantage of growing the phototrophs in an open system is that it is cheaper to operate than closed systems since there is no need for sterilization equipment, and the investment and maintenance costs are generally lower due to cheaper construction.

Alternatively the phototrophic community may be grown in a closed system. Suitable closed systems may include flat-panel reactors, tubular reactors and any other closed reactor design. The systems may be supplied with natural or artificial light as an energy source.

The basis for the medium is typically a water source. Suitable water sources include natural water sources such as lakes, rivers and oceans; and waste water sources such as municipal and industrial waste water. The temperature of the medium is typically 10-40° C., preferably 15-25° C. The pH of the medium is about 4-10, preferably about 6-9.

A further advantage of the methods of the present invention is that it may be coupled with flue gas CO₂ mitigation produced by power stations and waste water treatment. Waste water (e.g. sewage) may be pretreated for removal of organic carbon, while maintaining the essential nutrient concentrations required for cultivation of phototrophs. The pretreated waste water and the carbon dioxide may be used in the methods of the present invention. CO₂ produced from power stations or incineration installations may be used as a source of CO₂ in the methods of the present invention. Also CO₂ from the atmosphere may be used.

The phototrophic biomass may be collected by using conventional methods, such as microscreens, centrifugation, flocculation, broth flotation, ultrasound and combinations thereof.

The storage compound may be extracted from the biomass by existing technology (either destructive or non-destructive means. Such extraction processes may include physical extraction methods including crushing, pressing, osmotic shock and ultrasonication; and chemical extraction methods including solvent, enzymatic and supercritical carbon dioxide extraction).

There are numerous methods of converting the storage compound into a biofuel which are dependent on the type of storage compounds produced and the desired biofuel to be produced. Biofuels may include biodiesel, bio-ethanol, biogas, bio-hydrogen, bio-oil and bio-syngas. Preferably the biofuel is biodiesel or bio-ethanol. Biodiesel production utilizes a transesterification process, wherein the storage compounds, preferably lipids, undergo an alkali or acid catalyzed transesterification reaction. Glycerol is released as a byproduct of transesterification and fatty acid methyl esters are produced. This process may be run in either continuous or batch mode.

Bio-ethanol is naturally produced by some phototrophs and may be collected by non-destructive means without killing the microorganisms. The ethanol can be evaporated and subsequently condensed and collected. Alternatively, bio-ethanol may be produced by the action of microorganisms and enzymes through the fermentation of storage compounds, preferably polysaccharides such as glycogen or starch.

The methods of the present invention may be operated as a continuous process, but also as a sequenced batch.

The present invention is now elucidated on the basis of some examples.

### EXAMPLES

The influence of the selective pressure on the growth and storage compound production rates of phototrophic cultures was evaluated using an experimental reactor in which light energy and nitrogen-source supplies were separated and a control reactor in which light energy and nitrogen-source were supplied at the same time. Both reactors were inoculated with an amount of water from the Schie canal (Delft, NL), effectively containing hundreds of phototrophic species. A COMBO medium was used, which did not contain nitrogen-source so that this nutrient could be dosed separately and be depleted if desired. Cycle time was 24 hours, comprising 12 hours of light followed by 12 hours of darkness.

Growth was measured in terms of increase protein containing active biomass determined by TKN (Total Kjeldahl Nitrogen) and ammonium measurements and assuming an elemental composition of CH_{1.8}O_{0.5}N_{0.2}. Storage compounds production was measured by subtracting active biomass growth from the total increase in organic solids. These results were subsequently verified by GC and HPLC measurements.

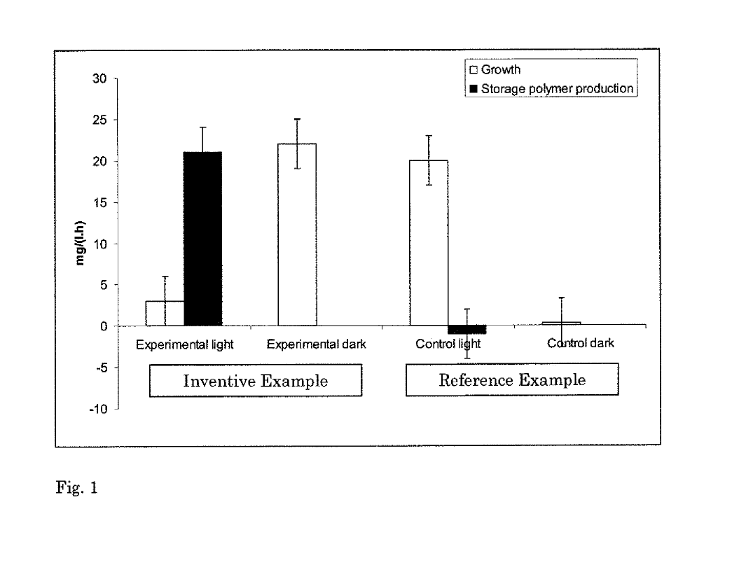

In FIG. 1 the results (in terms of storage compound production in mg/dm³·h) of a comparison of growth and storage compound production rates of algae in the light and dark phases of a dynamic operated reactor and a control bioreactor are shown. The nitrogen source was depleted in the light phase in the experimental reactor, but replenished in the dark phase. FIG. 1 clearly shows that storage compound production occurred in the experimental reactor during the light phase. The storage compound production in the control reactor was significantly less than in the experimental reactor (see FIG. 1). The growth of the algae was also significantly different in the reactors. In the experimental reactor the majority of algal growth occurs in the dark phase, while in the control reactor the majority of algal growth occurs in the light phase (see FIG. 1).

1. Method for producing an open phototrophic culture with improved storage compound production capability, comprising subjecting a starting culture to selective pressure, thus giving a competitive advantage to storage compound producing species, by subjecting said starting culture to a cycle of alternating dark phases and light phases and providing limitation of availability of essential growth nutrients in one or more of said light phases.

2. Method according to claim 1, wherein said open phototropic culture has a concentration of storage compounds of at least up to 50 wt. %, preferably at least up to 70 wt. % and more preferably up to 80-90 wt. % based on the weight of the dried cell mass.

3. Method according to any of claim 1, wherein an increase in storage compounds of more than 40% compared to the weight of storage compounds present in said starting culture is obtained within 7 hours after subjecting said starting culture to said cycle of alternating dark phases.

4. Method for producing microbial storage compounds comprising the steps of
selectively growing an open culture of a phototrophic community in a medium comprising nutrients during a dark phase, wherein the phototrophic community comprises one or more phototrophic species, and wherein the cell number of phototrophic species that grow on storage polymers in the dark increases;
creating unavailablility of one or more nutrients in the medium in a following light phase to prevent growth and induce the accumulation of storage polymers in the phototrophs; and
separating said storage compounds from said open culture.

5. Method according to claim 1, wherein the dark phase is 2-72 hours and essentially no light source is present.

6. Method according to claim 1, wherein the light phase is 2-72 hours and a light source is present.

7. Method according to claim 1, wherein the nutrients include at least one component selected from nitrogen, sulfur, molybdenum, magnesium, phosphorus, cobalt, nickel, silicon, zinc, copper, potassium, calcium, boron, chlorine, sodium, selenium, specific vitamins, and iron; and preferably all of said components.

8. Method according to claim 1, wherein the one or more nutrients depleted is selected from the group of nitrogen, iron, phosphorus and magnesium.

9. Method according to claim 1, wherein the storage compound includes lipids, polysaccharides, polyhydroxyalkanoates, and other carbon-based polymers.

10. Method according to claim 1, wherein the phototrophs include unicellular and multicellular photosynthetic microorganisms.

11. Method according to claim 1, wherein the phototrophs comprise algae.

12. Method according to claim 1, wherein said open phototrophic culture is non-axenic.

13. Method according to claim 4, wherein the dark phase is 2-72 hours and essentially no light source is present.

14. Method according to claim 4, wherein the light phase is 2-72 hours and a light source is present.

15. Method according to claim 4 , wherein the nutrients include at least one component selected from nitrogen, sulfur, molybdenum, magnesium, phosphorus, cobalt, nickel, silicon, zinc, copper, potassium, calcium, boron, chlorine, sodium, selenium, specific vitamins, and iron; and preferably all of said components.

16. Method according to claim 4, wherein the one or more nutrients depleted is selected from the group of nitrogen, iron, phosphorus and magnesium.

17. Method according to claim 4, wherein the storage compound includes lipids, polysaccharides, polyhydroxyalkanoates, and other carbon-based polymers.

18. Method according to claim 4, wherein the phototrophs include unicellular and multicellular photosynthetic microorganisms.

19. Method according to-claim 4, wherein the phototrophs comprise algae.

20. Method according to claim 4, wherein said open phototrophic culture is non-axenic.
